(19) United States
(12) Patent Application Publication
(10) Pub. No.: US 2008/0057108 A1
(43) Pub. Date: Mar. 6, 2008

(54) FUNCTIONAL FOOD AND METHOD FOR PREPARATION THEREOF

(75) Inventors: Antoine Koyazounda, Montmeyran (FR); Jean-Claude Millet, Etoile sur Rhone (FR)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: A'GOZZ NUTRITION SARL, Loriol sur Drome (FR)

(21) Appl. No.: 11/569,647

(22) Filed: Nov. 27, 2006

Related U.S. Application Data
(63) Continuation of application No. PCT/FR2005/001309, filed on May 27, 2005.

(30) Foreign Application Priority Data
May 27, 2004 (FR) 0405710

(51) Int. Cl. A61K 31/733; A23L 1/09; A23L 1/30; A61P 43/00; A61K 47/00; A23L 1/305

(52) U.S. Cl. 424/439; 426/302; 426/416; 426/431; 426/464; 426/506; 426/658; 426/71; 514/54

(57) ABSTRACT

A method is provided for preparing a functional food comprising the steps of: producing an aqueous solution of a malvaceous plant extract, adding fructan to the solution, and adjusting the solution to a determined temperature to obtain a solution containing fructose and/or oligofructose hydrolyzed from the fructan in the presence of the malvaceous plant extract. A functional food can be obtained from the method, which can be used to treat pathologies relating to calcium deficiency.

### CROSS-REFERENCE TO RELATED APPLICATIONS

This application is a Continuation of International Application No. PCT/FR2005/001309, filed May 27, 2005, which was published in the French language on Dec. 15, 2005, under International Publication No. WO 2005/117613 A1 and the disclosure of which is incorporated herein by reference.

### BACKGROUND OF THE INVENTION

The present invention relates to a functional food containing feuctose and/or oligofructose resulting from the hydrolysis of a fructan and/or fructan and to the method for preparation thereof. It relates more particularly to such a food in which the fructan is inulin.

Fructans are fructose polymers. Inulin is a fructan. It is formed by repitition of n units of fructose linked to one another by a −(2>1) glycosidic linkage, where n is between 2 and 70, and generally comprises a glucose molecule at the chain end because it is synthesized, in plants, using the sucrose.

Neither the enzymes of the human digestive system, nor the gastric acid, can break down the −(2>1) glycosidic linkage between the units of fructose. The inulin and the oligofructose, the units of which are linked by such a linkage, thus arrive intact in the colon where they undergo fermentation by the bacteria of the intestinal flora. Thus, the insulin enables this flora to be rebalanced. It has a periodic effect particularlyby its influence on the growth of lactobacilli and bifidobacteria. Furthermore, it contributes to the absorption of calcium and would probably contribute to reducing the level of cholesterol. Finally, it would have the property of repressing the production of a carcinogenic enzyme.

Inulin is marketed as is, in the form of dietary fibers, or, after chemical or enzymatic hydrolysis, in the form of fructose and glucose syrups in relative proportions of approximately 85% fructose to 15% glucose. The advantage of fructose lies in its very high sweetening power. It is mainly used in dietetic foods. Very widespread in its natural state, and particularly in fruit, pure fructose is difficult to produce industrially due to its instability at high temperatures and to its high solubility in water, which is an obstacle to crystallization. Its sweetening power at low temperature is 1.2 to 1.4 times greater than that of sucrose, but lower at high temperature. Its calorific value, on the order of 4 kcCal/g, is similar to that of sucrose and glucose.

Fructose syrups were included in the Common Market Organization for sugar in 1992 on the model of isoglucose. Their production quota is 0.3 MT for three Member States of the European Community. Nevertheless, production thereof has not experienced the development expected and is currently stagnating at only a little more than half of the quota provided.

Indeed, the methods of breaking down inulin are highly energy intensive, which increases the cost relating to the production of fructose.

In practice, these methods comprise several steps, the first of which are similar to those in the production of sugar. The inulin is extracted from the root of chicory which naturally contains, like beetroot with sugar, between 15 and 17% by weight inulin. Inulin is extracted by counter-current diffusion in hot water by means of a method similar to that in sugar production. Then, by a partial enzymatic hydrolysis method, the inulin is broken down into oligofructose, which has between 2 and 7 molecules of fructose. The oligofructose is separated into fractions of determined length ranging up to a single molecule of carbohydrate. These fractions constitute different products whose properties differ according to their molecular weight. The fructose syrups are prepared using these products, which can also be concentrated and, in certain cases, dried and atomized so as to form a powder.

### BRIEF SUMMARY OF THE INVENTION

In view of the state of the art that has just been described, a problem that the present invention intends to solve is that of producing a functional food containing fructose and/or oligofructose resulting from the hydrolysis of a fructan and/or fructan, which has inproved properties compared to the natural properties of inulin, and which is easier to prepare.

The object is achieved by a method for preparing a functional food, comprising the following steps: producing an aqueous solution of a malvaceous plant extract, adding fructan to the solution, and taking the solution to a determined temperature so as to obtain a solution containing fructose and/or oligofructose by hydrolysing the fructan in the presence of the malvaceous plant extract.

According to one embodiment, the solution of the malvaceous plant extract is produced by infusion or decoction followed by maceration of an extract of this plant.

According to one embodiment, the solution of the malvaceous plant extract, to which the fructan has been added, is dehydrated with a view to obtaining a residue.

According to one embodiment, the residue is nebulized.

According to one embodiment, the solution is transformed into a paste.

According to one embodiment, the paste is coated to obtain a dietetic confectionary.

According to one embodiment, the temperature of hydrolyzing the fructan is between approximately 70 and 110° C.

According to one embodiment, the fructan is inulin.

According to one embodiment, the malvaceous plant extract is an extract of calyxes of Hibiscus sabdariffa.

The present invention also relates to a functional food produced according to the method of the present invention, obtaining fructose and/or oligofructose resulting from the hydrolysis of a fructan and/or fructan, and a malvaceous plant extract.

According to one embodiment, the functional food contains between approximately 10 and 50% by weight of fructose, oligofructose and/or fructan, and between approximately 0.5 and 10% by weight of dried malvaceous plant extract.

According to one embodiment, the functional food forms a syrup or a liquid concentrate.

According to one embodiment, the functional food forms a soluble powder or a paste.

According to one embodiment, the functional food further contains polyunsaturated fatty acids.

According to one embodiment, the functional food contains calcium and anthocyanins and has a prebiotic effect.

According to one embodiment, the functional food is intended for the treatment of pathologies relating to a calcium deficiency.

The malvaceous plant extract itself has beneficial effects on the body. The fructan and the fructose or the oligofructose resulting from the hydrolysis of the fructan reinforce the intrinsic beneficial effects of the plant extract and, reciprocally, the preparation of the functional food does not require any step of enzymatic or chemical hydrolysis.

### DETAILED DESCRIPTION OF THE INVENTION

The present invention will be better understood in the light of the following description of examples of implementation of the present invention, which is not restrictive.

The functional food according to the present invention contains fructose, oligofructose and/or fructan in an amount of between approximately 10 and 50% by weight. The fructose and the oligofructose result from the breakdown of a fructan by hydrolysis. the fructan is advantageously inulin. In this case, the units of fructose of the oligofructose are linked by a −(2>1) glycosidic linkage which, in substance, is not broken down by the enzymes and the gastric acids of the human digestive system, and the oligofructose obtained by breakdown of the inulin is partly fructo-oligosaccharides having a terminal unit of glucose.

The functional food according to the present invention further contains a malvaceous plant extract in an amount of between approximately 0.5 and 10% by weight of dried plant extract.

The Malvaceous plants from which the extract according to the present invention is derived are advantageously Hibiscuses and, in particular, Hibiscus sabdariffa. There are more than 300 varieties of Hibiscus in the world. The Hibiscus sabdariffa is an annual and biannual herbaceous plant grown in tropical and subtropical regions. It can reach a height of 1.5 m. Its stems are robust, and are red or green depending on the variety. The leaves are oval, alternate, trifoliate or simple on the flowered stems. Its flowered are characterized by a calyx with 5 sepals, whose red or green color matches that of the stem. The fruits are capsular and, when ripe, the remaining calyx becomes fleshy. It measures 3 to 4 cm in length and 2 to 3 cm in width.

The calyxes of Hibiscus sabdariffa are particularly rich in anthocyanins, whose anti-oxidant properties for the body are well known. They are also rich in calcium, in vitamin C and, to a lesser extent, in iron and B vitamins.

The functional food thus has a high calcium content, the assimilation of this calcium by the body, which comes from the calyxes of Hibiscus sabdariffa, being helped by the inulin or its degradation products, such that this food is useful for the treatment of pathologies relating to a calcium deficiency, such as osteoporosis. The prebiotic effects of the functional food according to the present invention, which come essentially from the inulin, are not lessened in the presence of the calyxes of Hibiscus sabdariffa. The anti-oxidant effects of the inulin or of its degradation products are increased by the presence of the anthocyanins of Hibiscus sabdariffa. Finally, the food remains dietetic, particularly in that it helps to lower the level of cholesterol in the body depending on the type of inulin and in that its sweetening power, depending on its concentration in fructose, can be high without its calorific value necessarily being high. there is therefore a varitable synergy between the two components of the food according to the present invention, both of which are of plant origin.

The functional food according to the present invention can be prepared in syrup or liquid concentrate form, or in soluble powder or paste form. The hydrolysis of the glycosidic linkages linking the molecules of fructose of the inulin, is surprisingly performed at temperatures of between approximately 70 and 110° C., and solely in the presence of the malvaceous extract, whereas, without this extract, even at such temperatures, the inulin is not broken down.

### EXAMPLE 1

### Preparation in Liquid Concentrate Form

First, a liquid is produced by infusion or decoction followed by a maceration of 12 hours or more, without exceeding 24 hours, of 33 grams of dried red Hibiscus sabdariffa calyxes in one liter of water.

This liquid is sterilized by ebullition then put into containers, after filtration. A quantity of inulin in then added depending on the desired functional effect (e.g., perbiotic effect, calcium fixation, reduction in the absorption of cjolesterol), and they are mixed until a concentrated solution is obtained, that is put into a container preferably made of tinted glass to limit the effects of light and air. It is important not to simultaneously place the inulin and the calyxes in solution, otherwise the moxture precipitates and produces a solid mass which must then be ground. This solution is simply used by adding the desired quantity, such as one tablespoonful, into a container, which is then completed with hot or cold water. The solution is adjusted to a determined temperature, preferably between approximately 70 and 110° C., so as to obtain a solution containing fructose and/or oligofructose by hydrolyzing the inulin in the presence of the plant extract. Furthermore, a natural flavoring can be added by leaving the calyxes to infuse in the presence of fresh leaves, such as basil or mint for example, or thin slices of ginger.

### EXAMPLE 2

### Preparation in Soluble Powder Form, to Obtain a Drink or Capsules

In the same way as in Example 1, a liquid is first produced by infusion or decoction followed by a maceration of 12 hours or more, without exceeding 24 hours, of 33 grams of dried red Hibiscus sabdariffa calyxes in one liter of water. This liquid is then brought to a boil, first to be sterilized and second to obtain a sweet-tasting solution containing fructose and/or oligofructose resulting from the hydrolysis of the inulin, then is put into containers, after filtration. Then, at ambient temperature, 1,500 grams of this liquid are poured onto 1,000 grams of inulin powder in a container preferably made of glass. A paste is obtained which could then be used as it is. However, in the present example, this paste is spread on racks in relatively thin layers and passed through a drying tunnel or any other equivalent means. The drying conditions are such that the temperature of the paste is above 30° C. and never exceeds 38° C. Once the paste is dehydrated, a dry red residue is obtained.

In a first alternative embodiment, the dry red residue is transformed into powder by grinding. This red powder is fully soluble in hot water. It is therefore possible to prepare a cold or hot drink, such as a tisane, which has a combination of the properties of each element and benefits from their synergy.

In a second alternative embodiment, the dry extract is diluted in water until the mixture has a sufficiently low viscosity to be nebulized. This nebulization, performed in a hot atmosphere, makes it possible to return to a dry powder, which can be conditioned as capsules.

### EXAMPLE 3

### Preparation in Paste Form

In a first alternative, the paste obtained in Example 2 is dried. Then, first, another ingredient is added to it, such as a sesame paste to provide polyunsaturated fatty acids and particularly such acids of the omega 3 series (eicosapentaenoic acid and docosahexaenoic acid) and, second, a flavoring is added. This paste is then coated, for example with a thin layer of chocolate, to obtain an excellent dietetic confectionary.

In a second alternative, a tertiary infusion is prepared either hot or cold. For this purpose, dried Hibiscus sabdariffa calyxes are poured into water, in a proporttion of 33 g per 1,000 g of liquid. The mixture obtained is left to macerate for 24 hours in a sealed container. The tertiary infusion liquid is finally recovered through a strainer. This liquid is poured onto a powdered inulin without the mixture being stirred, and the whole result is placed in an oven pre-heated to 100° C. After exactly 30 minutes, the mixture becomes sweet and the sweetening power increases over the exposure time in proportion to the concentration of fructose and fructo-oligosaccharides (FOS) in the mixture, which come from the hydrolysis of the inulin by heat in the presence of the Hibiscus sabdariffa extract. The sweet mixture obtained is irreversibly liquid: it is no longer possible to obtain retrogradation into a solid product. This syrup with a variable concentration of fructose, depending on the duration of exposure of the mixture of heat, can be directly placed in opaque glass bottles to preserve the quality of the anthocyanins, and in the absense of air. It can no longer be atomized according to the classic industrial methods.

The preparation method thus enables a non-enzymatic hydrolysis of the inulin to be obtained at much lower costs than those of the current state of the art. It should be noted, however, that it is possible to obtain a paste consisting of a mixture of fructo-oligosaccharides (FOS) and fructose. In the same way as in the first alternative, this paste can be combined with another paste, a sesame paste for example, which adds flavor and polyunsuturated fatty acids particularly of omega 3 type. This other paste forms a basis for a chocolate-coated confectionary, for example, which has all the listed properties of the paste obtained according to the first alternative, and which furthermore is sweet, with all the benefits of fructose.

### EXAMPLE 4

### Preparation in Syrup Form

The preparation method described with regard to the second alternative of Example 3, pursued through to completion, leads to obtaining a liquid syrup, which can no longer be returned to a dry form and which, according to the analysis thereof, contains approximately 37% by weight of pure fructose.

In the example above, the composition of the mixture can be modified depending on the desired effects. Furthermore, the recommended daily intakes are adjusted according to the benefits sought.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. A method for preparing a functional food, the method comprising the following steps:
preparing a first aqueous solution of a malvaceous plant extract,
adding fructan to the first solution to a determined temperature to obtain a third solution containing fructose and/or oligofructose hydrolyzed from fructan in the presence of the malvaceous plant extract.

2. The method according to claim 1, wherein the first solution of the malvaceous plant extract is prepared by infusion or decoction followed by maceration of an extract of the plant.

3. The method according to claim 1, wherein the third solution is dehydrated to obtain a residue.

4. The method according to claim 3, wherein the residue is nebulized.

5. The method according to claim 1, wherein the third solution is transformed into a paste.

6. The method according to claim 5, wherein the paste is coated to obtain a dietetic confectionary.

7. The method according to claim 1, wherein the temperature of hydrolyzing the fructan is between 70 and 110° C.

8. The method according to claim 1, wherein the fructan is inulin.

9. The method according to claim 1, wherein the malvaceous plant extract is extract of calyxes of Hibiscus sabdariffa.

10. A functional food produced according to the method of claim 1, the functional food comprising a malvaceous plant extract and at least one of fructose, fructan, and oligofructose resulting from hydrolysis of a fructan.

11. The functional food according to claim 10, comprising between approximately 10 and 50% by weight of fructose, oligofructose and/or fructan, and between approximately 0.5 and 10% by weight of dried malvaceous plant extract.

12. The functional food according to claim 10, in a form of a syrup or a liquid concentrate.

13. The functional food according to claim 10,in a form of a soluble powder or a paste.

14. The functional food according to claim 10, further comprising polyunsaturated fatty acids.

15. The functional food according to claim 10, further comprising calcium and anthocyanins and having a prebiotic effect.

16. A method for treatment of pathologies relating to a calcium deficiency, the mehthod comprising administering to a subject suffering from the pathology the functional food according to claim 10.
